(19) United States
(12) Patent Application Publication
(10) Pub. No.: US 2004/0116864 A1
(43) Pub. Date: Jun. 17, 2004

(54) Catheter introducer assembly having safety shielded needle before and after use

(76) Inventor: Chad P. Boudreaux, Cincinnati, OH (US)

Correspondence Address: WOOD, HERRON & EVANS, LLP, 2700 CAREW TOWER, 441 VINE STREET, CINCINNATI, OH, 45202, US

(21) Appl. No.: 10/318,270

(22) Filed: Dec. 12, 2002

(51) Int. Cl. A61M 5/178

(52) U.S. Cl. 604/164.01; 604/164.12

(57) ABSTRACT

A catheter assembly for insertion into a vessel of a patient. The catheter assembly includes an elongate hollow tubular catheter having a distal end and a proximal end. The proximal end of the catheter has a hub attached thereto. The catheter assembly further includes a needle assembly having an elongated needle with a sharpened distal end and a proximal end. The needle is disposed within the catheter prior to insertion into a patient with the distal end of the needle being distal to the distal end of the catheter. The catheter assembly also includes a safety assembly. The safety assembly has a first position for protecting the sharpened distal end of the needle, and a second position for exposing the sharpened distal end of the needle. The safety assembly normally being in the first position and being movable to the second position for insertion into the vessel of a patient.

### FIELD OF THE INVENTION

The present invention relates, in general, to intravenous (IV) catheters and, more particularly, to a safety IV catheter with a needle tip protector that will protect the clinician from the needle tip before, during, and after use.

### BACKGROUND OF THE INVENTION

An intravenous (IV) catheter is an instrument that is used to introduce certain fluids such as saline solution directly into the bloodstream of a patient. Typically, a needle or other stylet is first introduced through the cannula portion of the catheter and into the skin of the patient at the desired location such as the back of the patient's hand or a vessel on the inside of the arm. Once insertion is complete, the needle is removed from the cannula portion of the catheter. After removing the needle, a fluid handling device such as a syringe is attached to the luer fitting located at the proximal end of the catheter hub. Fluid then flows directly from the fluid handling device through the catheter into the bloodstream of the patient.

Before the needle is introduced through the catheter and into the skin of the patient, a health care worker prepares the IV for use. The needle tip is exposed in this reparation creating a danger of an accidental needle stick to the health care worker which may cause injury. After preparation, the health care worker proceeds to insert the catheter into the vein of the patient. During insertion, the patient may suddenly move after the catheter is inserted into the vein, but before the needle tip protector is activated leaving the needle tip exposed. In addition, when the needle is removed from the cannula after insertion is complete, the health care worker must place the exposed needle tip at a nearby location while simultaneously addressing the task required to accomplish the needle removal. An exposed needle tip during insertion and removal creates a danger of an accidental needle stick occurring which leaves the health care worker vulnerable to the transmission of various, dangerous blood-borne pathogens such as human immune virus (HIV) and hepatitis.

The risk of a contaminated needle stick is not just isolated to the health care worker inserting the intravenous catheter. Careless disposal of used needles can put other health care workers at risk as well. Even others outside the health care profession, for example those involved in the clean-up and final disposal of medical waste, are at risk of an accidental needle stick from a carelessly discarded needle.

The danger to health care workers and others outside the health care profession from accidental needle sticks has yielded the development of catheters with safety mechanisms in which the occurrence of such accidental needle sticks is prevented. An example of a catheter having a safety mechanism is disclosed in U.S. Pat. No. Re. 34,416 issued to Lemieux. A safety catheter is described which includes an element that covers the needle tip upon removal of the needle from the catheter. The safety element includes a split flange at its proximal end which is expanded by the needle as the needle is inserted into an undersized hole at the center of this flange. The safety element is thus held secure within the catheter hub by inserting the needle through the undersized hole which forces the outside perimeter of the split flange against the inside wall of the catheter hub. One of the drawbacks to this design is the amount of friction force exerted against the needle by the split flange. A tight fit of the flange against the catheter wall causes great friction against the needle making it difficult to be withdrawn from the catheter by the clinician. A lose fit leaves the flange prone to releasing prematurely from the catheter as the needle is withdrawn, creating the potential that the needle tip will be left exposed. Another drawback to this design is that the needle is not protected before and during the insertion of the catheter into the patient creating the risk of an accidental needle stick.

Another example of a catheter having a safety mechanism is disclosed in U.S. Pat. No. 4,828,547 issued to Sahi et al. A self-blunting needle assembly is described comprising a hollow needle, which may be of conventional construction having a needle mouth and a needle shank terminating in a puncture tip. The hollow needle further includes a blunting member mounted therein including an elongate probe which slidably fits within the bore of the needle shank. The probe terminates in a distal tip which is initially positioned short of the puncture tip of the needle so as not to interfere with injection of the needle. After the injection is complete and during needle withdrawal, the blunting member is advanced to an extended position in which its distal tip protrudes beyond the puncture tip thereby blunting the needle. One drawback of this design is that the blunting member does not protect the needle tip prior to needle insertion. In addition, if during insertion, the health care worker slips and the needle is prematurely removed prior to completing insertion, the blunt does not extend past the needle tip protecting the health care worker from an accidental needle stick.

The prior art safety catheters all exhibit one or more drawbacks that have thus far limited their usefulness and full acceptance by health-care workers. None of the prior art safety catheters protect the health care clinician during the entire procedure: before the needle is inserted into the patient, during insertion in case of an accidental removal, and during the removal of the needle assembly. What is needed therefore is a safety IV catheter that functions reliably, is easy and inexpensive to manufacture, easy to use, and protects the needle tip throughout its use.

### SUMMARY OF THE INVENTION

A catheter assembly for insertion into a vessel of a patient. The catheter assembly includes an elongate hollow tubular catheter having a distal end and a proximal end. The proximal end of the catheter has a hub attached thereto. The catheter assembly further includes a needle assembly having an elongated needle with a sharpened distal end and a proximal end. The needle is disposed within the catheter prior to insertion into a patient with the distal end of the needle being distal to the distal end of the catheter. The catheter assembly also includes a safety assembly. The safety assembly has a first position for protecting the sharpened distal end of the needle, and a second position for exposing the sharpened distal end of the needle. The safety assembly normally being in the first position and being movable to the second position for insertion into the vessel of a patient.

### BRIEF DESCRIPTION OF THE DRAWINGS

The novel features of the invention are set forth with particularity in the appended claims. The invention itself, however, both as to organization and methods of operation, together with further objects and advantages thereof, may best be understood by reference to the following description, taken in conjunction with the accompanying drawings in which:

FIG. 1 is a perspective view of the IV catheter assembly of the present invention.

FIG. 2 is an exploded view of the IV catheter assembly of the present invention illustrating the elements therein.

FIG. 3 is a top view of the IV catheter assembly of the present invention illustrating the blunt extended beyond the needle tip when the buttons are released.

FIG. 4 is perspective view of the distal end of IV catheter assembly of the present invention illustrating the blunt extending beyond the needle tip.

FIG. 5 is a top view of the IV catheter assembly of the present invention illustrating the blunt retracted within the needle when the buttons are depressed.

FIG. 6 is perspective view of the distal end of IV catheter assembly of the present invention illustrating the blunt extending beyond the needle tip.

FIG. 7 is a top view of the cam in the present invention.

FIG. 8 is a bottom view of the cam in the present invention.

FIG. 9 is a side view of the cam in the present invention.

FIG. 10 is a side view of the first button in the present invention.

FIG. 11 is a side view of the first button in the present invention.

FIG. 12 is a cross-section view of the device showing the needle assembly when the buttons are released.

FIG. 13 is a cross-section view of the device showing the needle assembly when the buttons are released.

FIG. 14 is a perspective view of the blunt assembly in the present invention.

FIG. 15 is a cross section view of the IV catheter assembly illustrating the blunt extending beyond the needle tip.

FIG. 16 is a cross section view of the IV catheter assembly illustrating the blunt retracted within the needle.

FIG. 17 is a perspective view of an alternate embodiment of the IV assembly of the present invention with the blunt extending beyond the needle tip and the buttons released.

FIG. 18 is a perspective view of an alternate embodiment of the IV assembly of the present invention with the blunt retracted within the needle and the buttons depressed.

FIG. 19 is a top view of an alternate embodiment of the present invention illustrating the blunt extending beyond the needle when the buttons are released.

FIG. 20 is a top view of an alternate embodiment of the present invention illustrating the blunt retracted within the needle when the buttons are depressed.

FIG. 21 is an exploded view of an alternate embodiment of the present invention.

FIG. 22 is a top view of an alternate embodiment of the present invention.

FIG. 23 is a top view of an alternate embodiment of the present invention showing the blunt extending beyond the needle tip when the buttons are released.

FIG. 24 is a top view of an alternate embodiment of the present invention showing the blunt retracted within the needle when the buttons are depressed.

### DETAILED DESCRIPTION OF THE INVENTION

As used herein, the term “proximal” refers to a location on the catheter and needle assembly with needle tip protector closest to the clinician using the device and thus furthest from the patient on which the device is used. Conversely, the term “distal” refers to a location farthest from the clinician and closest to the patient.

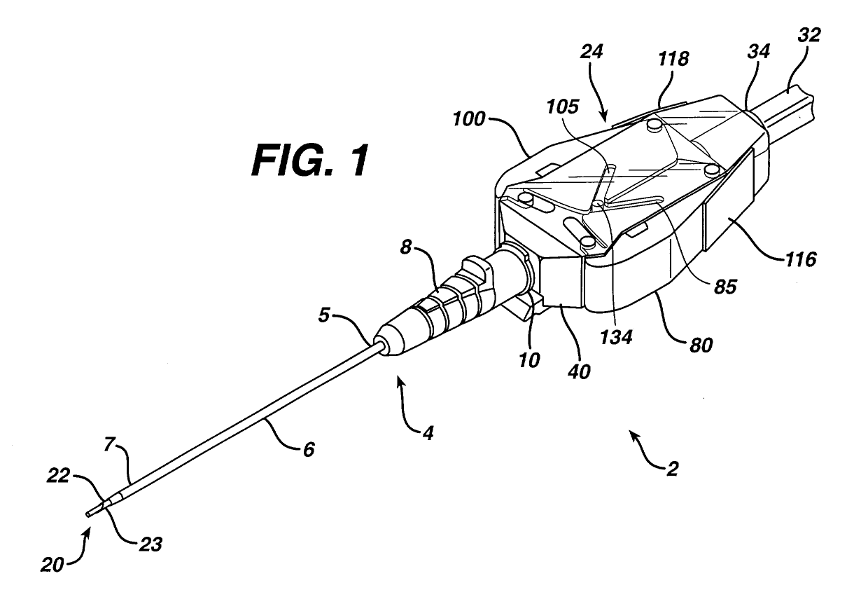

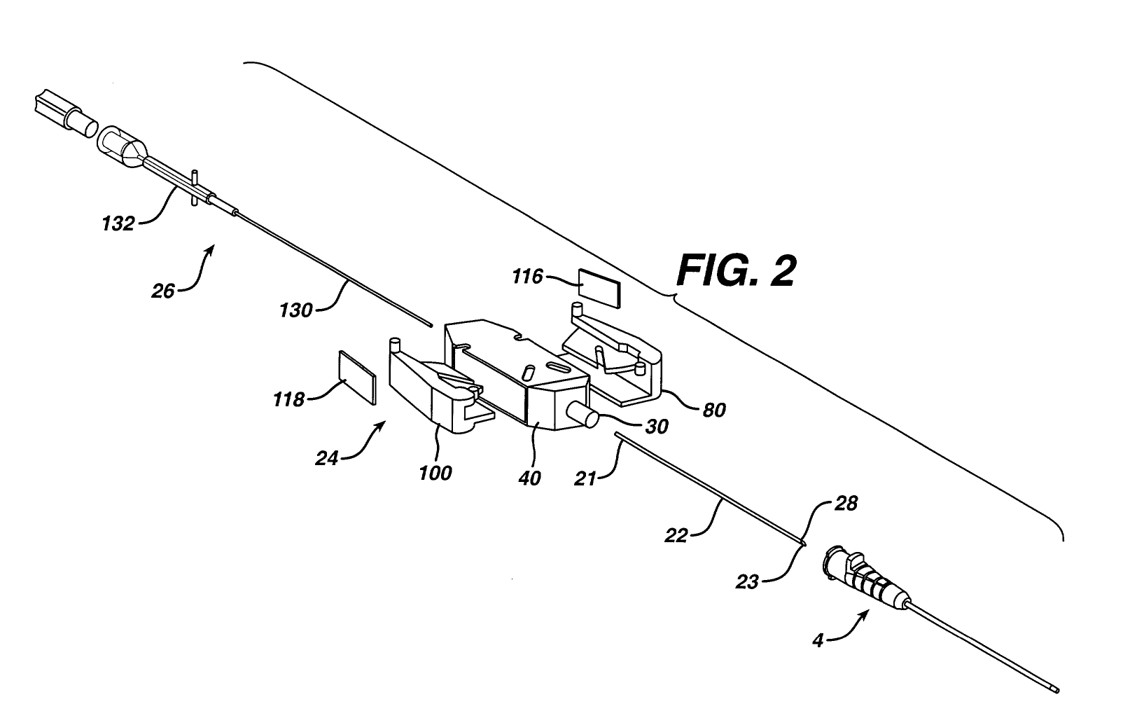

As illustrated in FIGS. 1 and 2, IV catheter assembly 2 comprises catheter assembly 4 and needle assembly 20. Needle assembly 20 further includes blunt or safety assembly 26. Catheter assembly 4 includes catheter 6 which is a tubular structure having a proximal end 5 and distal end 7. Proximal end 5 of catheter 6 is fixedly attached to catheter hub 8. Catheters are well known in the medical art and one of many suitable materials, most of which are flexible thermoplastics, may be selected for use in catheter 6. Such materials may include, for example, polyurethane or fluorinated ethylene propylene. Catheter hub 8 is a generally tubular structure having an internal cavity in fluid communication with the internal lumen of catheter 6. Catheter hub 8 may be made from a suitable, rigid medical grade thermoplastic such as, for example, polypropylene or polycarbonate. For illustration purposes catheter hub 8 is shown translucent, though in actual use it may be translucent or opaque. At the proximal end of catheter hub 8 is integrally attached Luer fitting 10, commonly known in the medical art. Luer fitting 10 provides for secure, leakproof attachment of tubing, syringes, or any of many other medical devices used to infuse or withdraw fluids through catheter assembly 4.

Referring again to FIGS. 1-6, needle assembly 20 comprises needle 22, which is a tubular structure with proximal end 21 and distal end 23, needle hub 24, and blunt assembly 26. Needle 22 which is preferably made of stainless steel has a lumen therethrough created by its inner diameter. Bevel 28 which is located at distal end 23 of needle 22 creates a sharp piercing tip. Needle hub 24, which is generally a box structure having an internal cavity in fluid communication with the lumen in needle 22, includes needle holder 30. Needle holder 30, which is generally tubular, is integrally attached to the proximal end of cam 40. The inner surface of needle holder 30 created by its inner diameter is fixedly attached to the proximal end 21 of needle. Needle holder 30 is preferably made of a translucent or transparent generally rigid thermoplastic material such as, for example, polycarbonate. At the most proximal end of the internal cavity in needle hub 24 is fixedly attached porous plug 32. A flashback chamber 34 is created in the cavity distal to porous plug 32. Porous plug 32 contains a plurality of microscopic openings which are large enough to permit the passage of air and other gasses but small enough to prevent the passage of blood. Flashback chamber 34 fills with blood upon successful entry of the needle tip into the targeted vein, providing the clinician visual conformation of the correct placement of the needle.

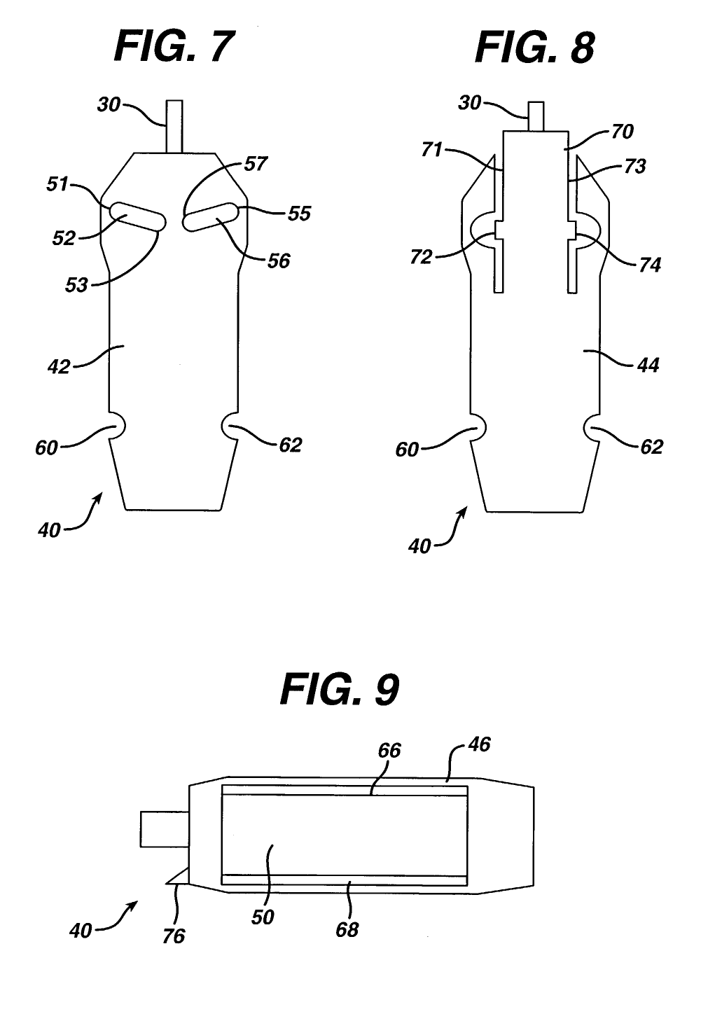

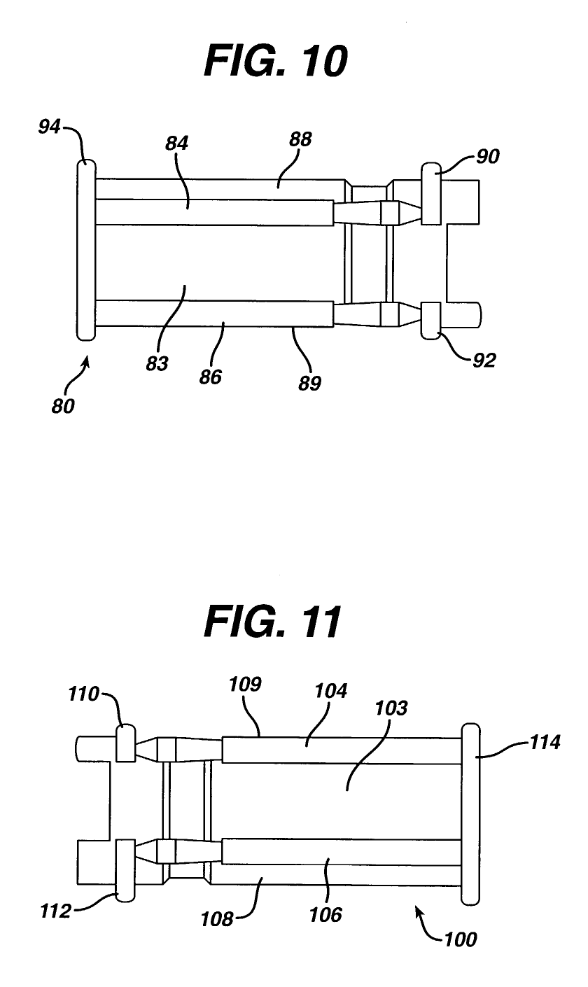

As shown in FIGS. 7-9, needle hub 24 further includes cam 40. Cam 40 is generally a box like structure preferably made of a rigid thermoplastic such as, for example, polycarbonate having top 42, bottom 44, first side 46, second side 48, and hollow 50 therethrough. Cam 40 further includes first eyelet 52, second eyelet 56, first hinge 60, second hinge 62, first channel 66, second channel 68, and cam arm 70. First eyelet 52, which is an opening on top 42 of cam 40, is generally oval shaped having first end 51 and second end 53. First eyelet 52, which is angled away from the longitudinal axis such that first end 51 is located above second end 53, is in fluid communication with hollow 50 of cam 40. Second eyelet 56, which is an opening on top 42 of cam 40, is generally oval shaped having first end 55 and second end 57. Second eyelet 56, which is angled away from the longitudinal axis such that first end 55 is located above second end 57, is in fluid communication with hollow 50 of cam 40. Cam 40 further includes first hinge 60 and second hinge 64. First hinge 60 is an arced notch in top 42, bottom 44, and first side 46 at the proximal end of cam 40. First hinge 60 allows first button 80 (shown in FIG. 10) to pivot in and out of cam 40 causing blunt 130 to extend and retract. Second hinge 62 is an arced notch in top 42, bottom 44, and second side 48 (not shown but located opposite of first side 46) at the proximal end of cam 40 opposite of first hinge 60. Second hinge 62 allows second button 100 (shown in FIG. 11) to pivot in and out of cam 40 causing blunt 130 to extend and retract. Top 42 of cam 40 includes first channel 66 therein. First channel 66 is a groove in top 42 which runs parallel to the longitudinal axis and plays an integral role in securing blunt 100 over the needle tip, which will be described in more detail later. Bottom 44 of cam 40 includes second channel 68 therein. Second channel 68 is a groove in bottom 44 which runs parallel to the longitudinal axis and plays an integral role in securing blunt 130 over the needle tip, which will be described in more detail later. Cam 40 further includes cam arm 70. Cam arm 70 is generally rectangular shaped having first cam lock 72 and second cam lock 74 extending laterally therefrom. Cam arm 70 is integrally molded from the middle to the distal end of bottom 44 of cam 40. Cam arm 70 further includes lip 76 integrally attached to its distal end. Lip 76 plays an important role in securing and releasing blunt assembly 26 so that it may cover and uncover the needle tip of needle 22. First cam lock 72, which is generally rectangular, is integrally attached to first wall 71 of cam arm 70 extending laterally therefrom. Second cam lock 74, which is generally rectangular, is integrally attached to second wall 73 of cam arm 70 extending laterally therefrom. First cam lock 72 and second cam lock 74, which play an important role in securing blunt 130 beyond the tip of needle 22 when first button 80 and second button 100 are released, will be described in more detail later. At the proximal end of cam 40 is an aperture therethrough. The aperture in cam 40 is in fluid communication with hollow 50 and helps blunt assembly 26 to move proximally and distally within cam 40.

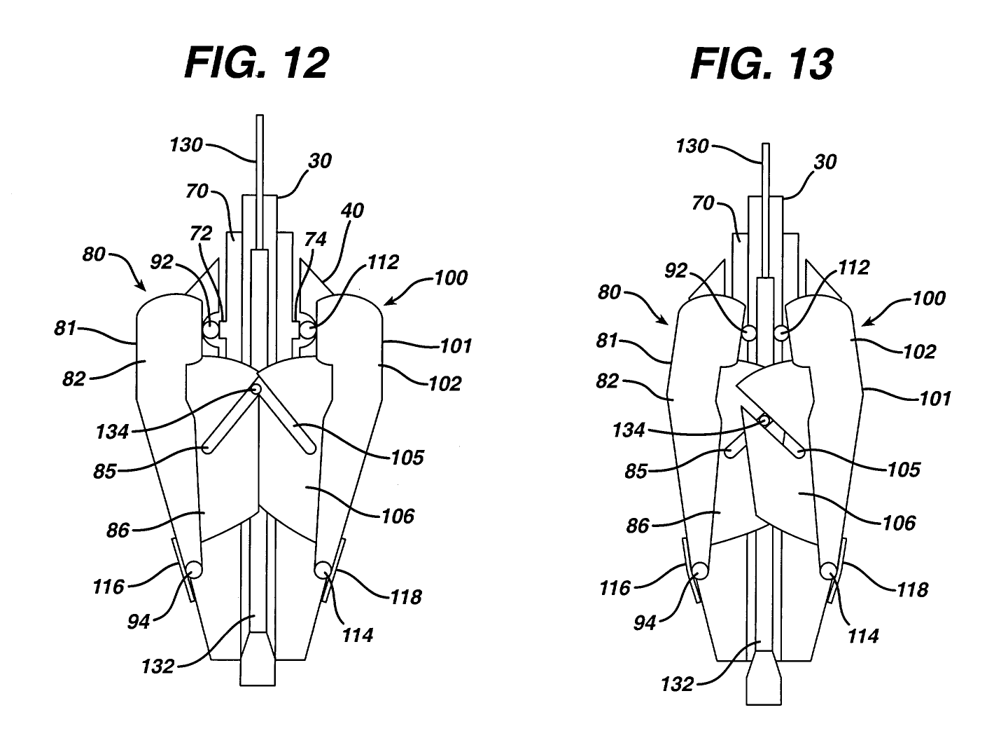

As shown in FIGS. 10-13, needle hub 24 further includes first button 80, second button 100, first spring 116, and second spring 118. First button 80, which is preferably made of a rigid thermoplastic such as, for example, polycarbonate, includes first button arm 82, first top wing 84, and first bottom wing 86. First button arm 82 is generally a pistol shape and includes first thumb press 81 and first arm wall 83. First arm wall 83 is integrally attached to first top wing 84 and first bottom wing 86 such that first top wing 84 is indented from the top of first button arm 82 forming first gap 88 and first bottom wing 86 is integrally attached to the bottom of first button arm 82 such that it forms first button surface 89. As shown in FIGS. 12 and 13, first top wing 84 has first top groove 85 extending diagonally therein. Similarly, first bottom wing 86 has first bottom groove 87 (not shown but directly under first top groove 85) extending diagonally therein. Integrally attached to the distal end of first button arm 82 is first button guide 90 and first blunt lock 92. First button guide 90, which is generally cylindrical, is integrally attached to the top of first arm wall 83 at the distal end of first button arm 82. First blunt lock 92, which is generally cylindrical, is integrally attached to the bottom of first arm wall 83 at the distal end of first button arm 82. Integrally attached to the proximal end of first button arm 82 is first button pin 94. First button pin 94, which is generally cylindrical, is preferably made of a rigid thermoplastic, such as, for example polycarbonate. Second button 100, which is preferably made of a rigid thermoplastic such as, for example, polycarbonate, includes second button arm 102, second top wing 104, and second bottom wing 106. Second button arm 102 is generally a pistol shape and includes second thumb press 101 and second arm wall 103. Second arm wall 103 is integrally attached to second top wing 104 and second bottom wing 106 such that second top wing 104 forms second button surface 109 and second bottom wing 106 forms second gap 108. As shown in FIGS. 12 and 13, second top wing 104 has second top groove 105 extending diagonally therein. Similarly, second bottom wing 106 has second bottom groove 107 (not shown but directly under second top groove 105) extending diagonally therein. Integrally attached to the distal end of second button arm 102 is second button guide 110 and second blunt lock 112. Second button guide 110, which is generally cylindrical, is integrally attached to the top of second arm wall 103 at the distal end of second button arm 102. Second blunt lock 112, which is generally cylindrical, is integrally attached to the bottom of second arm wall 103 at the distal end of second button arm 102. Integrally attached to the proximal end of second button arm 102 is second button pin 114. Second button pin 114, which is generally cylindrical, is preferably made of a rigid thermoplastic, such as, for example polycarbonate. First spring 116, which is preferably made of flexible polymer such as, for example, polyethylene is generally rectangular having first inner side 115 (not shown). First inner side 115 is fixedly attached to the proximal end of cam 40 and first button arm 82 such that half of first spring 116 resides on first button arm 82 and half resides on cam 40. Second spring 118, which is preferably made of flexible polymer such as, for example, polyethylene is generally rectangular having second inner side 117 (not shown). Second inner side 117 is fixedly attached to the proximal end of cam 40 and second button arm 102 such that half of second spring 118 resides on second button arm 102 and half resides on cam 40.

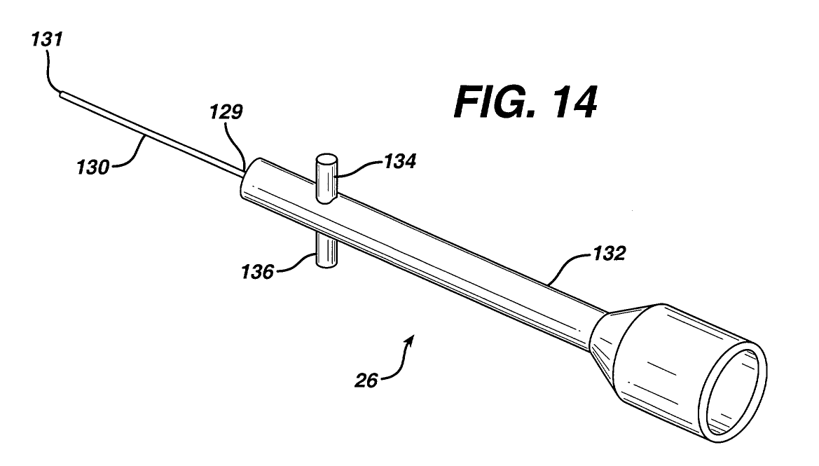

Referring now to FIG. 14, blunt assembly 26 includes blunt 130 and blunt holder 132. Blunt 130 has a proximal end 129 and a distal end 131 and is preferably a hollow tubular structure with a cavity therethrough formed from a single piece of thin, resilient material such as, for example, stainless steel or a polymer. Proximal end 129 of blunt 130 is fixedly attached to blunt holder 132. Blunt holder 132, which has a cavity therethrough in fluid communication with the cavity of blunt 130, is generally a tubular structure having a conical shaped distal end. Blunt holder 132 is preferably made of a translucent or transparent generally rigid thermoplastic material such as, for example, polycarbonate. Blunt assembly 26 further includes first channel pin 134 and second channel pin 136. First channel pin 134, which is generally cylindrical, is preferably made from a rigid thermoplastic such as, for example, polycarbonate. First channel pin 134 extends laterally from blunt holder 132 and plays an integral role in preventing blunt assembly 26 from moving axially in cam 40. Second channel pin 136, which is generally cylindrical, is preferably made from a rigid thermoplastic such as, for example, polycarbonate. Second channel pin 136 extends laterally from blunt holder 132 and plays an integral role in preventing blunt assembly 26 from moving axially in cam 40. Blunt assembly 26 plays an important role in protecting needle 22 and will be described in more detail later.

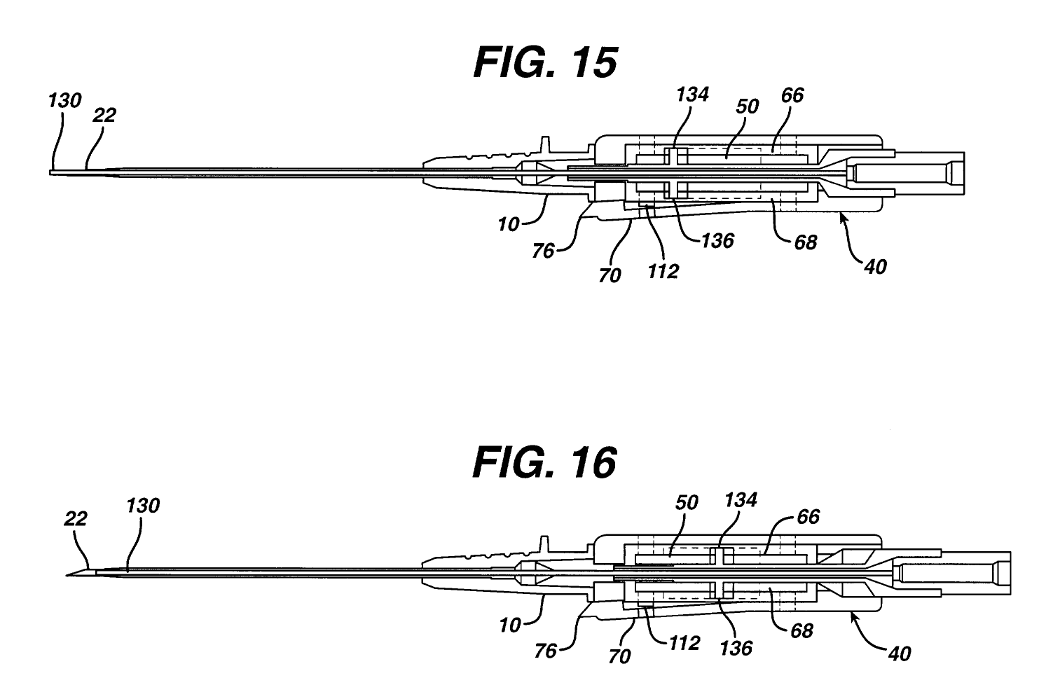

Referring now to FIGS. 15 and 16, it can understood how blunt assembly 26 is assembled to cam 40 and needle 22. The distal end of blunt 130 is inserted through the opening on the proximal end of cam 40 and then advanced through hollow 50 and into the lumen in needle 22, moving proximal to distal, until blunt 130 extends beyond the needle tip as shown in FIG. 15. During insertion of blunt 130, first channel pin 134 and second channel pin 136 are positioned such that first channel pin 134 is located in first channel 66 and second channel pin 136 is located in second channel 68 of cam 40. Placing first channel pin 134 in first channel 66 and second channel pin 136 in second channel 68 prevents axial movement of blunt assembly 26 in cam 40 during use.

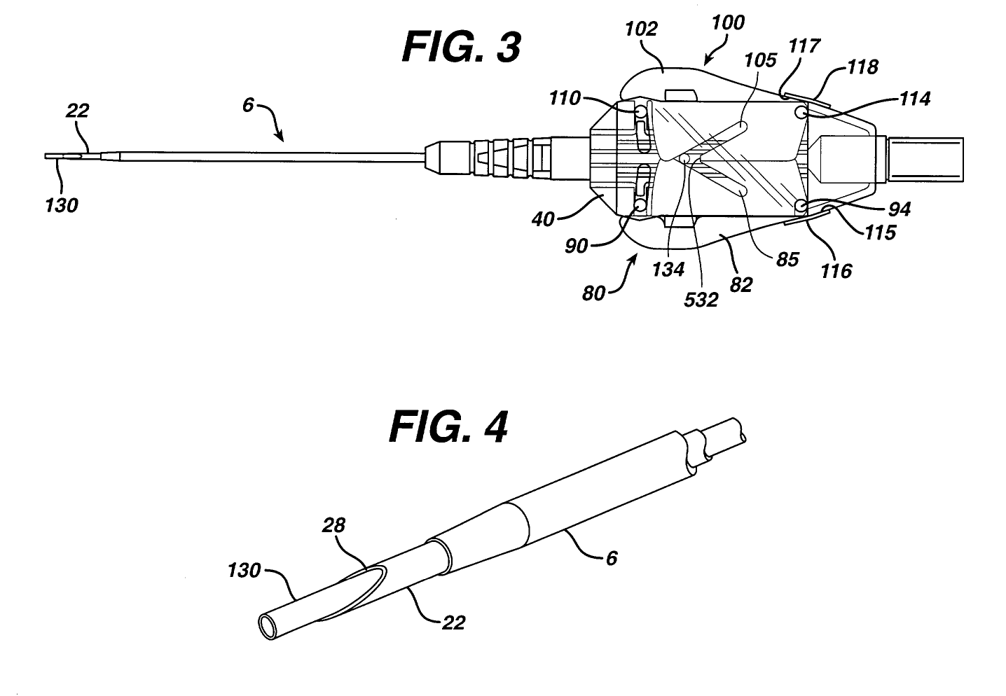

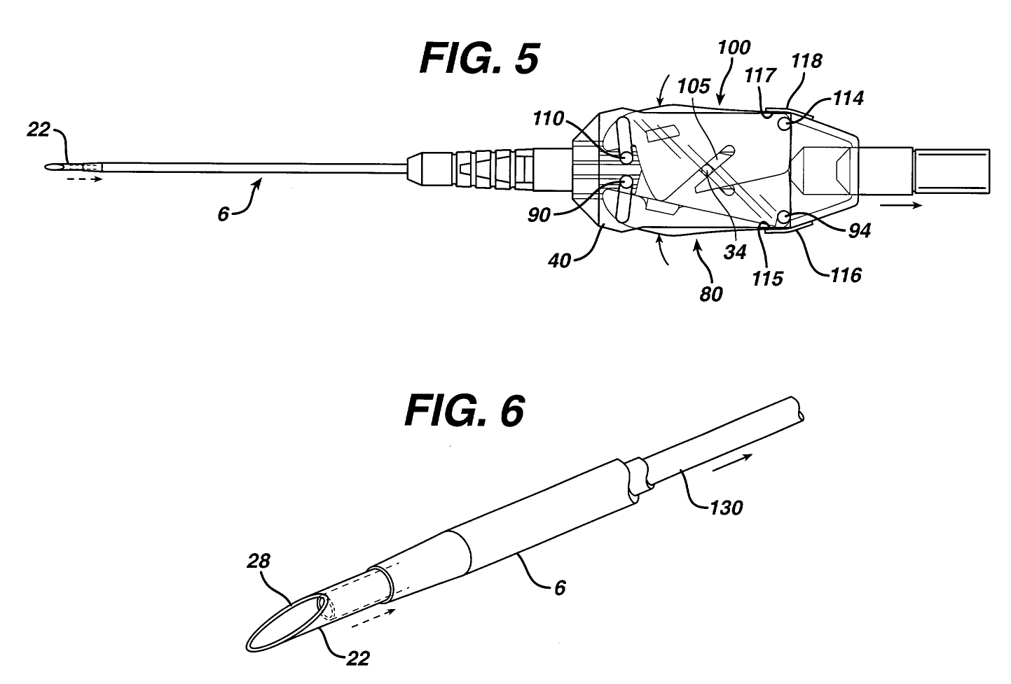

As shown in FIGS. 1-3, after blunt assembly 26 is assembled to cam 40 and needle 22, first button 80, second button 100, first spring 116, and second spring 118 are assembled to cam 40 to form needle assembly 20. First button pin 94 is snapped into first hinge 60 such that first button guide 90 resides in first eyelet 52 and first blunt lock 92 resides in first cam lock 72. Second button pin 114 is snapped into second hinge 68 such that second button guide 10 resides in second eyelet 56 and second blunt lock 112 resides in second cam lock 74. As shown in FIGS. 12 and 13, second top wing 104 is located above first top wing 84 and second bottom wing 106 is above first bottom wing 86 after first button 80 and second button 100 are connected to cam 40. In addition, first top groove 85 and second top groove 105 have first cannel pin 134 therebetween. Similarly, first bottom groove 87 and second bottom groove 107 have second cannel pin 136 therebetween. Having first channel pin 134 and second channel pin 136 in the grooves of the button wings creates a stopping mechanism which will not allow blunt assembly 26 from completely withdrawing from cam 40. First spring 116 can then be fixedly attached to first button 80 at the proximal end of cam 40. Similarly, second spring 118 can then be fixedly attached to second button 100 at the proximal end of cam 40. When first button 80 and second button 100 are biased outward by first spring 116 and second spring 118, as shown in FIGS. 3 and 4, blunt 130 extends beyond the needle tip in order to prevent a needle stick. Blunt 130 stays extended beyond the needle tip until first button 80 and second button 100 are depressed. Once depressed, first button 80 and second button 100 will retract blunt 130 behind the needle tip, as shown in FIGS. 5 and 6. Then when pressure is removed from first button 80 and second button 100, first spring 116 and second spring 118 will reset first button 80 and second button 100 to its biased outward position and blunt 130 will extend again to protect the needle tip.

As shown in FIG. 1, needle assembly 24, including blunt 130, is assembled into catheter assembly 4. Distal end 23 of needle 22, which has blunt 130 extending therefrom to protect the needle tip, extends distally from distal end 7 of catheter 6. Blunt 130 is retained past the needle tip by first blunt lock 92 locking in first cam lock 72 on cam arm 70 and second blunt lock 112 locking into second cam lock 74 on cam arm 70, while the distal end of needle 22 and blunt 130 is being inserted into catheter 6. Needle assembly 20 is secured onto luer fitting 10 of catheter hub 30 so that lip 76 is biased away from cam 40, which unlocks first button 80 and second button 100 allowing blunt 130 to be retracted within needle 22 once first button 80 and second button 100 are depressed.

Now, it will be described how in actual clinical use, the IV catheter assembly 2 of the present invention functions. The clinician begins by connecting the catheter assembly 4 to the needle assembly 20 such that luer fitting 10 is biased against lip 76 of cam 40. Securing luer fitting 10 such that it is biased against lip 72 causes cam arm 70 to be forced away from cam 40 releasing first cam lock 72 and second cam lock 74 from first blunt lock 92 and second blunt lock 112. Attaching the catheter assembly to the needle hub moves the blunt to a first position wherein the distal end of the blunt is distal to the distal end of the needle and enables the clinician to squeeze the buttons or actuator. The clinician then depresses and holds down first button 80 and second button 100, which pivot into cam 40. While first button 80 and second button 100 are being depressed, first top groove 85 and second top groove 105 and first bottom groove 87 and second groove 107, which have first channel pin 134 and second channel pin 136 residing therein respectively, force blunt assembly 26 proximally retracting blunt 130 within needle 22 as shown in FIG. 16. Depressing the buttons causes the blunt to move proximally to a second position, thereby exposing the needle tip. The distal end of needle 38 which extends just past the distal end of catheter 6 is then inserted into the patient's vein. After observing blood in flashback chamber 34, the clinician grasps needle hub 24, and catheter assembly 4 alone is moved distally into the vein. The clinician applies slight pressure to the insertion site to hold catheter assembly 4 secure. Then, the clinician grasps needle hub 24 and begins withdrawal of needle assembly 20 from catheter assembly 4. During this process, blunt 130 remains secure inside needle 22 until first button 80 and second button 100 are released. When first button 80 and second button 100 are released, first spring 116 and second spring 118 return first button 80 and second button 100 to its outward position causing first top groove 85 and second top groove 105 to move first channel pin 134 and first bottom groove 87 and second bottom groove 107 to move second channel pin 136 distally along with blunt assembly 26 as shown in FIG. 15. The distal movement of blunt assembly 26 causes blunt 130 to extend distally past the tip of needle 22. When the clinician releases the buttons, the blunt moves distally covering the needle tip. When first button 80 and second button 100 are completely released, first spring 116 and second spring 118 hold the buttons in place while the first blunt lock 92 and second blunt lock 112 secure into first cam lock 72 and second cam lock 74, since cam arm 70 has been released from its biased position on luer fitting 10 of catheter 6. Securing first blunt lock 92 and second blunt lock 112 into first cam lock 72 and second cam lock 74 prevents first button 80 and second button 100 from being depressed so that the needle tip is exposed. Needle assembly 20 is now removed entirely from catheter assembly 4, with the needle tip covered by blunt 130 of the present invention. Removing the catheter from the needle assembly causes the blunt to lock beyond the needle tip protecting the clinician from an accidental needle stick. In addition, if during the insertion of IV catheter assembly 2, the patient suddenly moves before the insertion is complete, the clinician can release first button 80 and second button 100. When first button 80 and second button 100 are released, first spring 116 and second spring 118 return first button 80 and second button 100 to its outward position causing first top groove 85 and second top groove 105 to move first channel pin 134 and first bottom groove 87 and second bottom groove 107 to move second channel pin 136 distally along with blunt assembly 26 as shown in FIG. 15. The distal movement of blunt assembly 26 causes blunt 130 to extend distally past the needle tip of needle 22 protecting the clinician during use of an accidental needle stick.

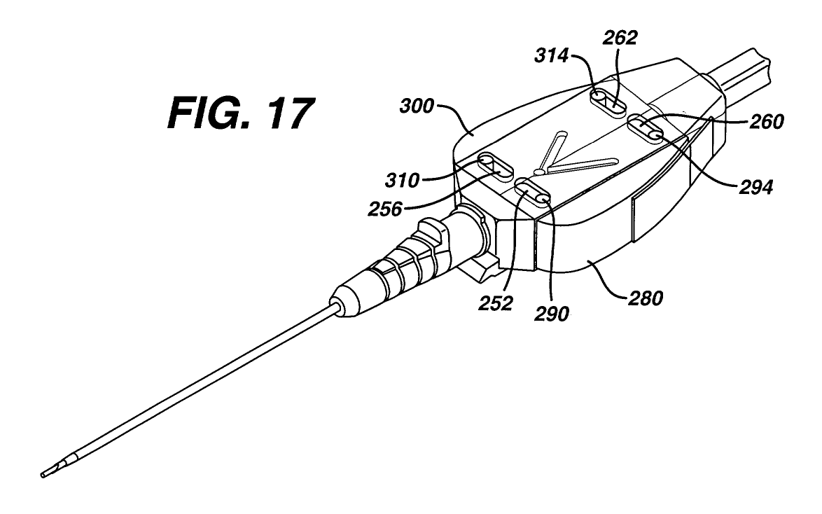

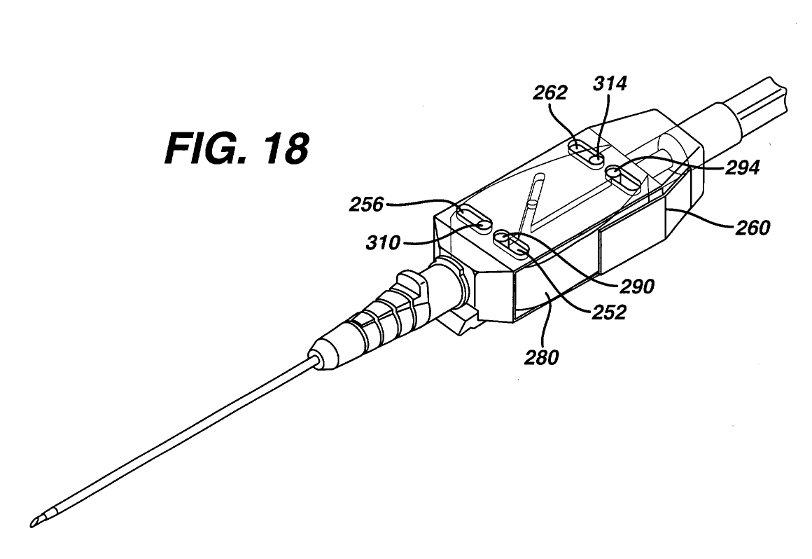

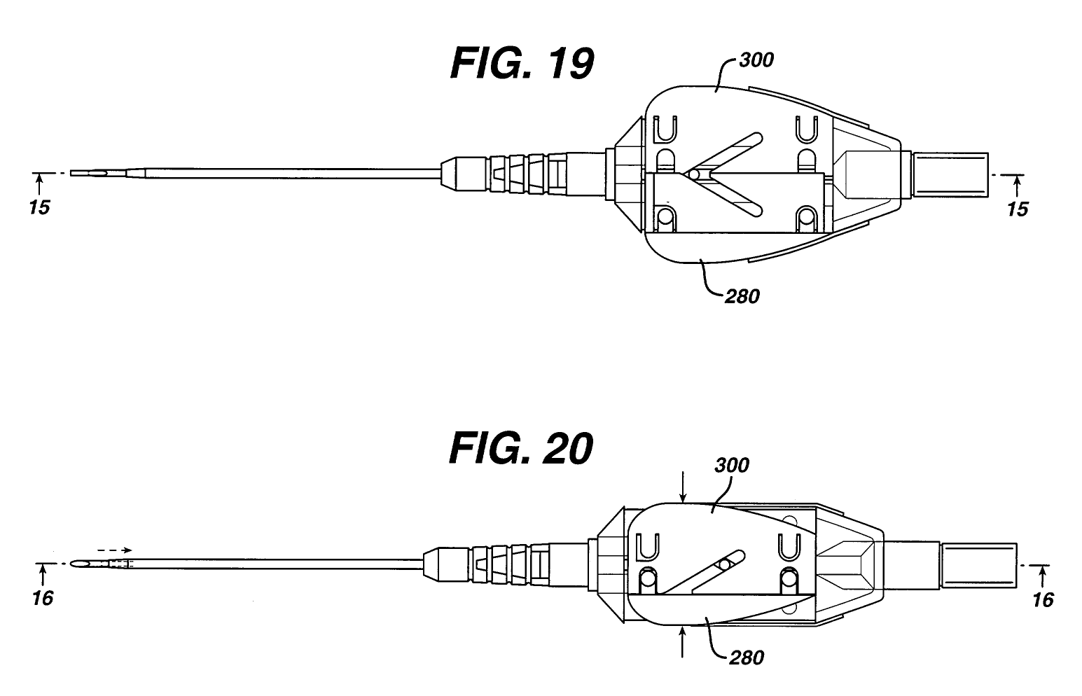

A first alternate embodiment of the present invention is shown in FIGS. 17-20. Needle assembly 220, similar to needle assembly 20, includes cam 240. Cam 240 includes first hinge slot 260 and second hinge slot 262 therein at its proximal end, which replace first hinge 60 and second hinge 62. First hinge slot 260, which extends perpendicular to the longitudinal axis of catheter assembly 2, is generally an oval shaped opening in top 242 and bottom 244 and includes first button pin 294 therein. Similarly, second hinge slot 262, which extends perpendicular to the longitudinal axis of catheter assembly 202, is generally an oval shaped opening in top 242 and bottom 244 and includes first button pin 314 therein. Cam 240 further includes first eyelet 252 and second eyelet 256 therein at its distal end. First eyelet 252 and second eyelet 256, similar to first eyelet 52 and second eyelet 56, are generally oval shaped openings in top 242 that extend perpendicular to the longitudinal axis of catheter assembly 202. First eyelet 252 has first button guide 290 of first button 280 residing therein. Similarly, second eyelet 256 has second button guide 310 of second button 300 residing therein. First hinge slot 260 and second hinge slot 262 allow first button 280 and second button 300 respectively to slide rather than pivot into cam 240.

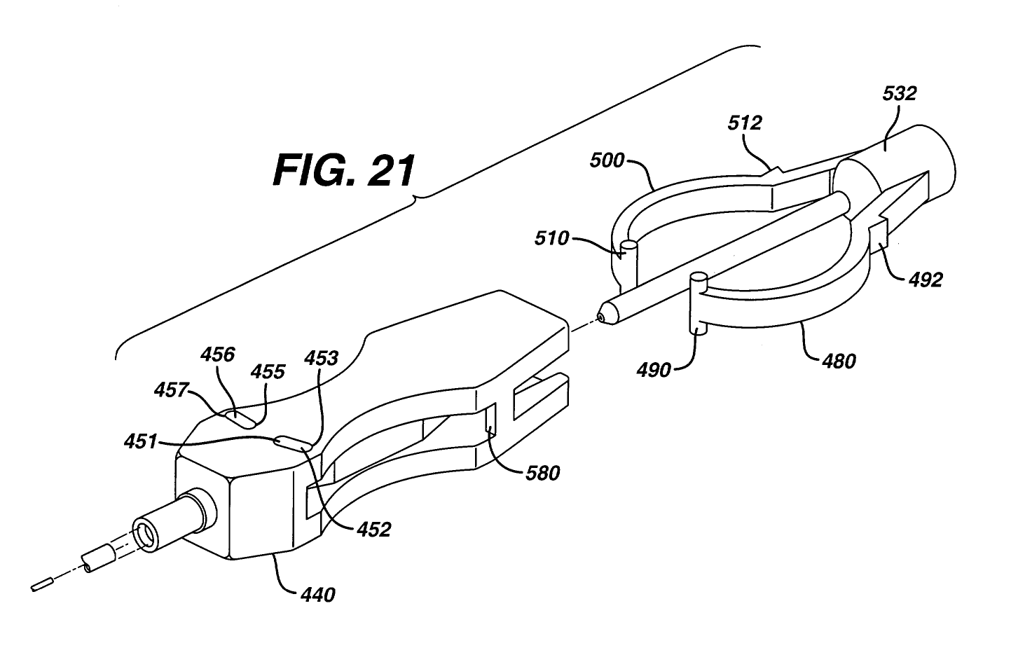

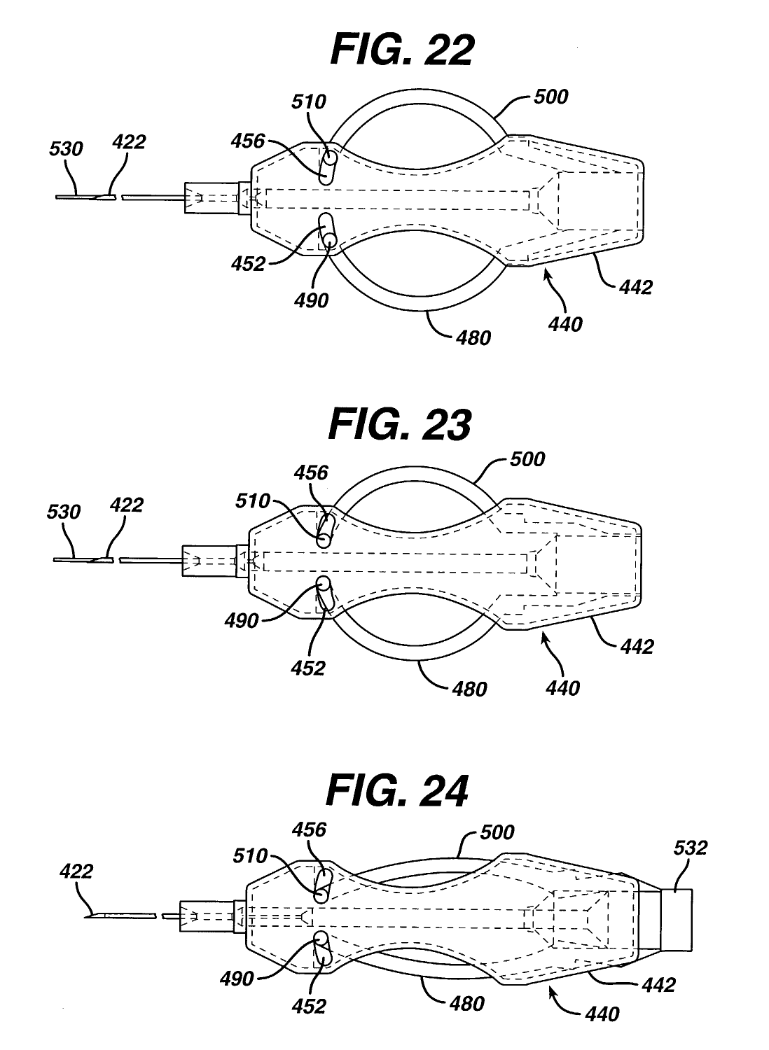

A second alternate embodiment of the present invention is shown in FIGS. 21-24. Needle assembly 220, similar to needle assembly 20, includes cam 440, first button 480, and second button 500. Cam 440 includes first cam stop 580 and second cam stop 582 at its proximal end. First cam stop 580 and second cam stop 582 are flat surfaces in cam 440 which prevents blunt 130 from retracting proximally and exposing needle 422 when first button 480 and second button 500 are released. Cam 440 further includes first eyelet 452 and second eyelet 456. First eyelet 452, which is an opening on top 442 and bottom 444 of cam 440, is generally oval shaped having first end 451 and second end 453. First eyelet 452, which is angled away from the longitudinal axis such that first end 451 is located above second end 453, is in fluid communication with hollow 450 of cam 440 and has first button pin 490 therein. Second eyelet 456, which is an opening on top 442 of cam 440, is generally oval shaped having first end 455 and second end 457. Second eyelet 456, which is angled away from the longitudinal axis such that first end 455 is located above second end 457, is in fluid communication with hollow 450 of cam 440 and has second button pin 510 therein. First button 480 is generally a semi-circular thin wireform preferably made of a spring-like plastic, such as, for example polycarbonate. Integrally attached to the distal end of first button 480 is first button guide 490. First button guide 490, which is generally cylindrical, resides in first eyelet 452 of cam 440 and helps guide first button 480 into cam 440 when it is depressed and released during the procedure. Integrally attached to the proximal end of first button 480 is first blunt lock 492. First blunt lock 492 is generally a rectangular block which helps secure blunt 530 over needle 422. Second button 500 is generally a semi-circular thin wireform preferably made of a spring-like plastic, such as, for example polycarbonate. Integrally attached to the distal end of second button 500 is second button guide 510. Second button guide 510, which is generally cylindrical, resides in second eyelet 456 of cam 440 and helps guide second button 500 into cam 440 when it is depressed and released during the procedure. Integrally attached to the proximal end of second button 510 is second blunt lock 512. Second blunt lock 512 is generally a rectangular block which helps secure blunt 530 over needle 422. The proximal ends of first button 480 and second button 500 are fixedly attached to the distal end of blunt holder 532 as shown in FIG. 21. When first button 480 and second button 500 are depressed, first blunt lock 492 and second blunt lock 512 are released from first blunt stop 580 and second blunt stop 582 respectively. After the release of first blunt lock 492 and second blunt lock 512, blunt 530 and blunt holder 532 are moved proximally exposing needle 422. When first button 480 and second button 500 are released, blunt 530 and blunt holder 532 slide distally so that blunt 530 extends beyond needle 122 protecting the user from an accidental needle stick.

While preferred embodiments of the present invention have been shown and described herein, it will be obvious to those skilled in the art that such embodiments are provided by way of example only. Numerous variations, changes, and substitutions will now occur to those skilled in the art without departing from the invention. For example, as would be apparent to those skilled in the art, the disclosures herein have equal application in robotic-assisted surgery. In addition, it should be understood that every structure described above has a function and such structure can be referred to as a means for performing that function. Accordingly, it is intended that the invention be limited only by the spirit and scope of the appended claims.

What is claimed is:

1. A catheter assembly for insertion into a vessel of a patient, said catheter assembly comprising:
a. an elongate hollow tubular catheter having a distal end and a proximal end, said proximal end of said catheter having a hub attached thereto;
b. a needle assembly comprising an elongated needle having a sharpened distal end and a proximal end, said needle is disposed within said catheter prior to insertion into a patient with said distal end of said needle being distal to said distal end of said catheter; and
c. a safety assembly having a first position for protecting said sharpened distal end of said needle, and a second position for exposing said sharpened distal end of said needle, said safety assembly normally being in said first position when said needle assembly is disposed within said catheter and being movable to said second position for insertion into the vessel of a patient.

2. The catheter assembly of claim 1 further including a handle attached to said proximal end of said needle assembly.

3. The catheter assembly of claim 2 wherein said handle includes an actuator disposed thereon for moving said safety assembly from said first position to said second position.

4. The catheter assembly of claim 3 wherein said actuator includes at least one cam for activating said actuator.

5. The catheter assembly of claim 1 wherein said safety assembly is moved to its first position upon inserting said needle assembly into said catheter.

6. A catheter assembly for insertion into a vessel of a patient, said catheter assembly comprising:
a. an elongate hollow tubular catheter having a distal end and a proximal end, said proximal end of said catheter having a hub attached thereto;
b. a needle assembly comprising an elongated hollow needle having a sharpened distal end a proximal end, said needle is disposed within said catheter prior to insertion into a patient with said distal end of said needle extending beyond the distal end of said catheter;
c. an elongated safety blunt having distal and proximal ends, said blunt being disposed within said hollow needle, said blunt having a first position wherein said distal end of said blunt extends beyond the distal end of said needle for protecting said sharpened distal end of said needle, and a second position wherein said distal end of said blunt is proximal to said distal end of said needle for exposing said sharpened distal end of said needle; and
d. an actuator for moving said blunt from said first position to said second position.

7. The catheter assembly of claim 5 further including a handle attached to said proximal end of said needle assembly.

8. The catheter assembly of claim 6 wherein said actuator is disposed on said handle.

9. The catheter assembly of claim 7 wherein said actuator includes at least one cam for activating said actuator.

10. The catheter assembly of claim 1 wherein said blunt is moved to its first position upon inserting said needle assembly into said catheter.
